(19) United States
(12) Patent Application Publication
(10) Pub. No.: US 2013/0317121 A1
(43) Pub. Date: Nov. 28, 2013

(54) METHOD FOR THE PREPARATION OF PLYMERS WITH VARIED ARCHTECTURE AND AMIDE INITIATION

(71) Applicants: Didier Bourissou, Plaisance Du Touch (FR); Blanca Martin-Vaca, Toulouse (FR); Aurélie Alba, Noueilles (FR); Roland Cherif-Cheikh, Castelldefels (Barcelona) (ES); Anne-Paula De Sousa Delgado, Molins de Rei (Barcelona) (ES)

(72) Inventors: Didier Bourissou, Plaisance Du Touch (FR); Blanca Martin-Vaca, Toulouse (FR); Aurélie Alba, Noueilles (FR); Roland Cherif-Cheikh, Castelldefels (Barcelona) (ES); Anne-Paula De Sousa Delgado, Molins de Rei (Barcelona) (ES)

(73) Assignee: IPSEN PHARMA S.A.S., Boulogne-Billancourt (FR)

(21) Appl. No.: 13/885,654

(22) PCT Filed: Nov. 14, 2011
(86) PCT No.: PCT/FR2011/000602

§ 371 Date: Jul. 30, 2013

(30) Foreign Application Priority Data
Nov. 15, 2010 (FR) 1004428

(51) Int. Cl. A61K 47/34

(52) U.S. Cl. CPC A61K 47/34
USPC 514/772.7; 560/180

(57) ABSTRACT

The invention relates to a method for preparing linear polymers having an amide end or having a star architecture comprising an amide core, by means of a ring opening using lactide and glycolide monomers or a lactide monomer ring in the presence of a catalyst, wherein the method includes the steps of: (i) reacting the excess monomer(s) with an initiator in a solvent, said initiator being selected from among an amine and an amino alcohol, given that the initiator has at least one primary or secondary amine function; (ii) adding a catalyst, said catalyst being a non-nucleophilic base and including at least one neutral sp2 nitrogen atom; and (iii) neutralizing the reaction mixture. Said novel method is particularly advantageous in that it can be easily monitored and enables better modulation of the polymers, and thus of the properties thereof, than the methods of the prior art. The invention also relates to novel polymers that are obtainable by means of said method.

### FIELD OF THE INVENTION

A subject of the present application is a method for the preparation of polymers with varied architectures (linear and star), based on lactide and/or glycolide, as well as novel polymers which can optionally be obtained by this method. These polymers have useful physico-chemical properties. This method can be easily controlled and offers a better adjustment of the polymers and therefore of their properties than the methods of the prior art.

### STATE OF THE ART

Nowadays, attention is increasingly being paid to synthetic polymers for preparing artificial organs and formulating medicaments [Chem. Eng. News 2001, 79 (6), 30]. The polymers concerned must satisfy a certain number of criteria and, in particular, they must be biocompatible. Biodegradability is an additional advantage if the polymer must be eliminated after an appropriate period of implantation in an organism. In this regard, copolymers based on lactic and glycolic acid (PLGA) are of very great benefit as they are sensitive to hydrolysis and are degraded in vivo with the release of non-toxic by-products. The field of application of the PLGAs is very broad (Adv. Mater. 1996, 8, 305 and Chemosphere 2001, 43, 49). In the field of surgery, they are used for the synthesis of multi-strand wires, sutures, implants, prostheses etc. In pharmacology, they allow the encapsulation, transfer and controlled release of active ingredients. For all these applications, one of the key factors is the degradation rate of the PLGAs which certainly depends on their structure (chain length, dispersity, proportion, stereochemistry and chain formation of the monomers etc.).

In order to obtain novel properties, it can be useful to modify the structure of the PLGA. However, the possible modifications are very limited and some have already been described: molar mass, tacticity etc. One of the parameters that has not been explored very much is modification of the ends. However, the physical properties and the degradation rates of PLGA with an ester end are different to those of a PLGA with an acid end have been described (WO200804963). In fact, a novel function could provide useful properties.

The applicant has noted that the PLGAs having an amide end could be particularly useful. Now, the majority of current methods do not take into account that the initiators of the hydroxy function (alcohol/water), make it possible to obtain PLGAs with an ester/acid end. An example of obtaining a polylactide structure (PLA) by nucleophilic catalysis of the polymerization of the lactide starting from an initiator of primary amine type (RNH₂), is described in O. Coulembier, M. K. Kiesewetter, A. Mason, P. Dubois, J. L. Hedrick, R. M. Waymouth, Angew. Chem. Int. Ed. 2007, 46, 4719. Starting from a poly(ethyleneglycol) functionalized with primary amines, ring opening polymerization (ROP) of the lactide catalyzed by carbenes gives access to PLAs with complex architectures. On each primary amine, two PLA arms grow. It is therefore not possible by this method to graft a single branch onto a primary amine. J. Liu, L. Liu, Macromolecules 2004, 37, 2674 describes obtaining a single linear polyester with an amide end. It is only described with polycaprolactones (PCL) (bulk polymerization for 24 to 48 hours at 160° C.).

Branched polymers, which include star polymers, dendrimers and hyperbranched polymers, have been the subject of numerous studies, due to their useful rheological and mechanical properties.

In particular, star polymers, or polymers with star architecture, can be used in the administration of active ingredients and have useful release profiles. This type of polymer is generally prepared from polyol initiators comprising n alcohol functions in order to produce stars with n arms.

Moreover, the star polymers have glass transition temperatures, as well as a viscosity in the vitreous state, slightly lower than their linear equivalents. The same applies as regards their crystallinity—and therefore their melting temperature—which is also lower than their linear equivalents. However, the crystalline phase retains the same nature in both architectures.

A biodegradable star polymer (for example, PLGA) will have a much more rapid initial degradation rate than its linear equivalent with the same mass. In fact, the release and degradation rate is to be correlated with the structure of the polymer matrix. It has been shown that by chemical or enzymatic hydrolysis, the first cleavages of ester bonds take place in the core of the star, close to the initiator, thus releasing linear polymers with lower molecular masses. On the other hand, an example of a star polymer with a PEG core and an amide-PLA bond where the first cleavages occur on the ester bonds and the amide bonds hydrolyze later (Biomacromolecules 2010, 11, 224).

These differences in properties therefore give access to useful innovative matrices. For example, the encapsulation of active ingredients in star polymers in the case of PLGAs has been described by A. Breitenbach, Y. X. Li, T. Kissel, Journal of Controlled Release 2000, 64, 167.

Ring-opening polymerization starting from metallic complexes for the synthesis of polymers with star architecture has been described since the 1990s. The star polymers are mainly prepared by solution or bulk polymerization, with metallic catalysts such as tin octanoate, even if other systems based on Fe, Zn, Al etc. have been reported (H. R. Kricheldorf, Polymer for Advanced Technologies 2002, 13, 969; A. Finne, A. -C. Albertsson, Biomacromolecules 2002, 3, 684; H. R. Kricheldorf, H. Hachmann-Thiessen, G. Schwarz, Biomacromolecules 2004, 5, 492; I. Arvanitoyannis, A. Nakayama, E. Psomiadou, N. Kawasaki, N. Yamamoto, Polymer 1996, 37, 651).

The applicant has developed a novel non-metallic method, which can be easily controlled and which has greater flexibility than the methods of the prior art.

The applicant has also developed new linear polymers with an amide end or with star architecture with an amide core.

### SUMMARY OF THE INVENTION

A subject of the invention is therefore a method for the preparation of linear polymers with an amide end or with star architecture with an amide core by ring opening based on lactide and glycolide monomers or a lactide monomer, comprising the steps consisting of:
(i) reacting the monomer or monomers in excess with an initiator in a solvent, said initiator being chosen from an amine and an amino alcohol, given that the initiator has at least one primary or secondary amine function,
(ii) adding a catalyst, said catalyst being a non-nucleophilic base, and comprising at least one nitrogen atom of sp2 type,
(iii) neutralizing the reaction mixture.

and preferentially the steps consisting of:
(iv) reacting the monomer or monomers in excess with an initiator in a solvent, said initiator being chosen from an amine and an amino alcohol, given that the initiator has at least one primary or secondary amine function,
(v) adding a catalyst, said catalyst being a non-nucleophilic base, and comprising at least one neutral nitrogen atom of sp2 type,
(vi) neutralizing the reaction mixture.

Preferably, the monomer is lactide.

Preferably, the polymers are prepared based on a lactide monomer and a glycolide monomer.

Preferably, step (ii) is carried out after the complete incorporation of the initiator.

Preferably, the basic catalyst is chosen from:

1,8-diazabicyclo[5.4.0]undec-7-ene (DBU),

1,5-diazabicyclo[4.3.0]non-5-ene (DBN),

a 4-amino-pyridine compound of formula:

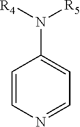

in which R₄ and R₅ are independently chosen from a hydrogen atom or a C₁-C₁₂ alkyl radical; or R₄ and R₅ form together with the nitrogen atom which bears them a saturated heterocycle;

a cyclic guanidine of formula:

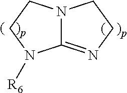

in which p is 1 or 2, and R₆ represents a hydrogen atom or a C₁-C₄ alkyl radical;

a phosphazene of formula:

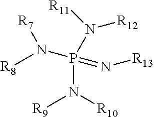

in which R7, R10, R11, and R12 represent independently a C1 to C6 alkyl radical,

R₈ and R₉ represent independently a hydrogen atom or a C1 to C6 alkyl radical, or R₃ and R₉ form together with the nitrogen atoms which bear them a saturated heterocycle,

R₁₃ represents a C1 to C6 alkyl radical.

Preferably, the reaction takes place in an organic solvent, preferably in a halogenated or aromatic solvent.

Preferably, the solvent is a halogenated solvent, preferably, the solvent is dichloromethane.

Preferably, the initiator is an amine.

Preferably, the initiator is an amino alcohol.

Preferably, the reaction temperature is from 0 to 150° C., preferably from 20 to 45° C.

This method has the advantage of allowing the complete incorporation of the initiator into the polymer chains as an amide end and therefore leading to a very good initiation efficiency.

A subject of the invention is also novel polymers of formula I:

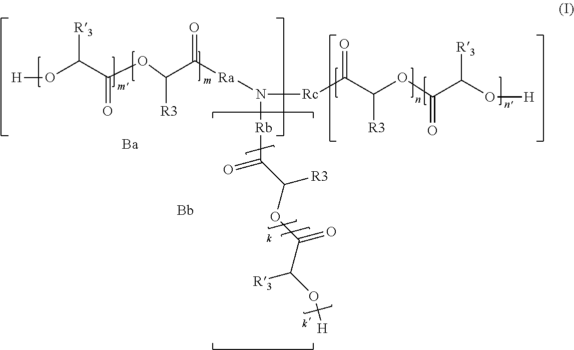

in which

n, n′, m, m′, k and k′ represent independently an integer from 0 to 12,

Ra represents a covalent bond, a linear or branched C5 to C14 alkyl radical, an alkylamino radical, an alkyloxy radical, an aryl or aralkyl radical, it being understood that if Ra is an aryl or aralkyl radical then m and m′ are zero,

Rb represents a covalent bond, a linear or branched C5 to C14 alkyl radical, an alkylamino radical or an alkyloxy radical,

Rc represents a covalent bond, a linear or branched C5 to C14 alkyl radical, an alkylamino radical, or an alkyloxy radical,

R3 represents a hydrogen atom and R′3 represents an alkyl radical, it being understood that at least one of n′, m′ and k′ is different from zero;

or R′3 represents a hydrogen atom and R3 represents an alkyl radical, it being understood that at least one of n, m and k is different from zero;

and it being understood that:

at most one of the branches Ba, Bb and Bc represents a hydrogen atom

if one of the branches Ba, Bb and Bc represents the hydrogen atom, then at least one of the other two branches is linked to the nitrogen atom by an alkylamino radical.

Preferably, R3 represents an alkyl radical, and n′, m′ and k′ are zero.

Preferably, one of the branches Ba, Bb and Bc represents the hydrogen atom.

Preferably, at least one of Ra, Rb and Rc represents an alkylamino radical.

Preferably, at least one of Ra, Rb and Rc represents an alkyloxy radical.

A subject of the invention is also a pharmaceutical composition comprising at least one polymer according to the invention.

### BRIEF DESCRIPTION OF THE FIGURES

FIG. 1 represents the electron microscope photograph of a polymer with a CO—NCH3-C12alkyl end (Example 8).

FIG. 2 represents the electron microscope photograph of a polymer with a CO—NH—C12 alkyl end (Example 1).

### DETAILED DESCRIPTION OF EMBODIMENTS OF THE INVENTION

Therefore a subject of the invention is a method for the preparation of linear polymers with an amide end or with star architecture with an amide core.

By star polymer, is meant a polymer having a single branch point from where several linear chains (branches) emanate. By “amide core”, is meant that the branch point is a nitrogen atom and that at least one of the linear chains comprises at least one other nitrogen atom (at the “core” of the polymer) linked to a —C(═O)— radical in order to form an amide function. On the linear chain or chains comprising the amide function, there are at most 10 successive atoms which separate the branch point nitrogen atom from the nitrogen atom of the amide function, preferably at most 5 atoms, more preferably at most 3 atoms, yet more preferably at most 2 atoms.

For example, the polymer

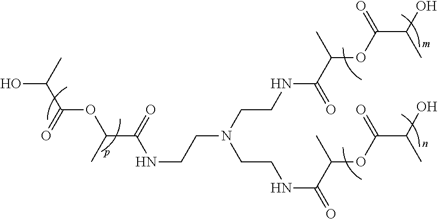

is a polymer with an amide core: the branch point form which three linear chains emanate is a nitrogen atom; two atoms separate the nitrogen atom of an amide function from the nitrogen atom branch point.

By linear polymers with an amide end, is meant a linear polymer having one of its two ends of non-substituted, N-monosubstituted or N,N-disubstituted amide type. For example, a linear polymer having a —C(═O)—NH—C₁₂H₂₅ end is meant.

The polymerization reaction is of ring-opening type. Ring-opening polymerization is an addition polymerization. It can be diagrammatically represented as follows:

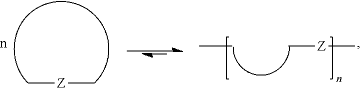

with n being the number of monomers.

The reaction is carried out starting from a lactide monomer and a glycolide monomer, or from a lactide monomer alone. According to a variant, the monomer is lactide. According to another variant, the reaction is a co-polymerization and the reaction is carried out starting from lactide and glycolide.

The method comprises a first step (i) consisting of reacting the monomer or monomers with an initiator in a solvent. The monomer or monomers must be in excess with respect to the initiator, preferably from 1/1 to 100/1, more preferably from 1/1 to 30/1, yet more preferably from 1/1 to 6/1.

The initiator is chosen from an amine and an amino alcohol.

By amine, is meant any compound comprising at least one primary, secondary or tertiary amine function. For example the alkylamines, diaminoalkyls or diaminoalkyls are meant. For example, tris(2-aminoethyl)amine is meant.

By aminoalcool, is meant any compound comprising at least one primary, secondary or tertiary amine function and at least one—OH function. For example, diethanolamine is meant.

It is understood that the initiator has at least one primary or secondary amine function.

The method comprises a second step (ii) consisting of adding a catalyst.

Preferably, step (ii) is carried out after all the initiator has been incorporated in step (i), i.e. no more initiator remains in the reaction mixture.

No more initiator remains in the reaction mixture when the reaction between the added initiator and the stoichiometric quantity of lactide is finished. The reaction can be monitored by proton NMR, and in this case, the catalyst is added when no more initiator signals are seen.

For example, step (ii) is carried out after a duration comprised between 5 and 30 minutes after the start of step (i), preferably, from 10 to 20 minutes.

This allows an excellent initiation efficiency to be obtained.

The catalyst is a non-nucleophilic base, preferably a non-nucleophilic strong base. The catalyst comprises at least one nitrogen atom of sp2 type, i.e. the nitrogen is of ═N— type, i.e. bound on the one side (to a first adjacent atom) by a double bond and on the other side (to a second adjacent atom) by a single bond. Preferably, the catalyst comprises at least one neutral nitrogen atom of sp2 type. The catalyst, non-nucleophilic base, preferably reacts as a Bronsted base and not as a nucleophile.

The catalyst is a non-nucleophilic base which can be chosen from the diazacycloalkene derivatives; the amino-pyridine derivatives such as the 4-amino-pyridine derivatives; the cyclic guanidine derivatives; or the phosphazene derivatives.

The catalyst is a non-nucleophilic base which can be preferentially chosen from:

the diazacycloalkene derivatives such as the diazabicycloundecenes and diazabicyclononenes;

the 4-amino-pyridine derivatives such as the 4-amino-pyridines derivatives of formula:

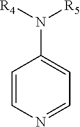

in which R4 and R5 are independently chosen from a hydrogen atom or a C1-C12 alkyl radical; or R4 and R5 form together with the nitrogen atom which bears them a saturated heterocycle;

the cyclic guanidine derivatives of formula

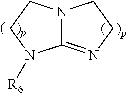

in which p is 1 or 2, and R6 represents a hydrogen atom or a C1-C4 alkyl radical;

or the phosphazene derivatives of formula:

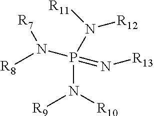

in which R7, R10, R11, and R12 represent independently a C1 to C6 alkyl radical,

R8 and R9 represent independently a hydrogen atom or a C1 to C6 alkyl radical, or R8 and R9 form together with the nitrogen atoms which bear them a saturated heterocycle,

R13 represents a C1 to C6 alkyl radical.

The catalyst is a non-nucleophilic base which can be preferentially chosen from: 1,8-diazabicyclo[5.4.0]undec-7-ene (or DBU), 1,5-diazabicyclo[4.3.0]non-5-ene (DBN); N′,N′-dimethylamino-4-pyridine (or DMAP), 1,5,7-triazabicyclo-[4.4.0]dec-5-ene (TBD), 2-tert-butylimino-2-diethylamino-1,3-dimethylperhydro-1,3,2-diazaphosphorine (or BEMP).

For example, the catalyst is DBU (1,8-diazabicyclo[5.4.0]undec-7-ene).

Preferably, the catalyst is a 4-amino-pyridine compound of formula:

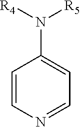

in which R4 and R5 are independently chosen from a hydrogen atom or a C1-C12 alkyl radical; or R4 and R5 form together with the nitrogen atom which bears them a saturated heterocycle. By a 4-amino-pyridine compound of formula:

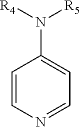

is meant for example N′,N′-dimethylamino-4-pyridine (or DMAP).

Preferably, the catalyst is a cyclic guanidine of formula:

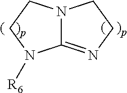

in which p is 1 or 2, and R6 represents a hydrogen atom or a C1-C4 alkyl radical. By cyclic guanidine, is meant for example 1,5,7-triazabicyclo-[4.4.0]dec-5-ene (TBD).

Preferably, the catalyst is a phosphazene, and preferentially a monophosphazene. Preferably, the catalyst is a monophosphazene of formula:

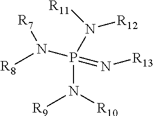

in which R7, R10, R11, and R12 represent independently a C1 to C6 alkyl radical,

R8 and R9 represent independently a hydrogen atom or a C1 to C6 alkyl radical, or R8 and R9 form together with the nitrogen atoms which bear them a saturated heterocycle,

R13 represents a C1 to C6 alkyl radical.

By a monophosphazene compound as defined above, is meant, for example 2-tert-butylimino-2-diethylamino-1,3-dimethylperhydro-1,3,2-diazaphosphorine (BEMP).

Preferably, the ratio of the initial concentration of the NH2 function of the initiator to the concentration of catalyst is from 1 to 1000, more preferably from 2 to 500, yet more preferably from 10 to 100.

The method comprises a third step (iii) consisting of neutralizing the reaction mixture. The neutralization can be carried out by any means known to a person skilled in the art. For example, the neutralization is carried out by the addition of an acid, or an acid resin such as Amberlyst™ A15.

The reaction takes place in a solvent. The term “solvent” here means a single solvent or a mixture of solvents. Preferably, the solvent is chosen so that the polymer formed is soluble therein. Preferably, the solvent is chosen from the halogenated solvents, the cyclic ethers and the aromatic solvents. For example, the solvent is chosen from dichloromethane, dichloroethane, tetrahydrofuran (THF) and toluene. Preferably, the solvent is dichloromethane.

Preferably, the reaction is carried out at a temperature comprised between ambient temperature, i.e. approximately 25° C., and the boiling temperature of the chosen solvent. The reaction temperature is chosen so as to be below the degradation temperature of the polymer formed. For example, the temperature is from 0 to 150° C. Preferably, the temperature is from 10 to 90° C. Preferably also, the temperature is from 20 to 45° C., preferably from 20 to 30° C. For example, the reaction is carried out at ambient temperature.

Alternatively, step (i) is carried out by heating, preferably at a temperature comprised between 50 and 80° C., preferably by heating to reflux. This is preferred when the initiator is a secondary amine.

In this alternative, step (ii) is preferably carried out at a temperature from 15 to 35° C., preferably, at ambient temperature.

Preferably, the reaction is stopped by step (iii) once the desired degree of polymerization is obtained. For example, the reaction is stopped when the consumption of initial monomer is from 90 to 100%. Preferably the reaction is stopped when the consumption of initial monomer initial is greater than 96%.

The method according to the invention has numerous advantages. In particular, the method allows high selectivity. This method has the advantage of allowing the complete incorporation of the initiator in the polymer chains as amide end and therefore leading to a very good initiator efficiency. Thanks to this method it is possible to obtain very varied polymers, having easily adjustable properties. It is possible to obtain a polymer with 1, 2 or 3 branches with an amide end.

Preferably, the polymer obtained is a polymer with one branch with an amide end. Preferably, the polymer obtained is a polymer with two branches with an amide end. Preferably, the polymer obtained is a polymer with three branches with an amide end.

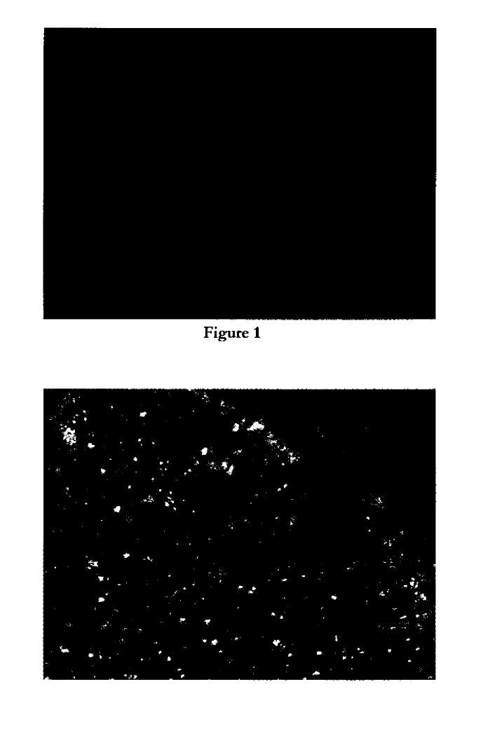

FIGS. 1 and 2 show these differences in properties.

FIG. 1 shows a photograph taken under polarized light using an electron microscope, of the polymer with a CO—NCH3-C12alkyl end (Example 8). The photograph, which is black, shows that the polymer is amorphous.

FIG. 2 shows a photograph taken under polarized light using an electron microscope, of the polymer with a CO—NH—C12alkyl end (Example 1). The polymer is crystalline.

The invention also relates to a novel polymer of formula I:

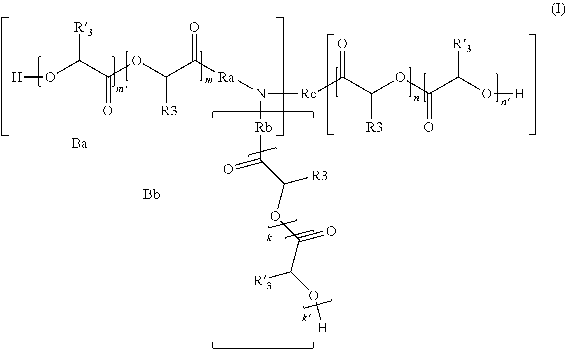

in which

n, n′, m, m′, k and k′ represent independently an integer from 0 to 12,

Ra represents a covalent bond, a linear or branched C5 to C14 alkyl radical, an alkylamino radical, an alkyloxy radical, an aryl or aralkyl radical, it being understood that if Ra is an aryl or aralkyl radical then m and m′ are zero,

Rb represents a covalent bond, a linear or branched C5 to C14 alkyl radical, an alkylamino radical or an alkyloxy radical,

Rc represents a covalent bond, a linear or branched C5 to C14 alkyl radical, an alkylamino radical, or an alkyloxy radical,

R3 represents a hydrogen atom and R′3 represents an alkyl radical, it being understood that at least one of n′, m′ and k′ is different from zero;

or R′3 represents a hydrogen atom and R3 represents an alkyl radical, it being understood that at least one of n, in and k is different from zero;

and it being understood that:

at most one of the branches Ba, Bb and Bc represents a hydrogen atom

if one of the branches Ba, Bb and Bc represents the hydrogen atom, then at least one of the other two branches is linked to the nitrogen atom by an alkylamino radical.

Unless defined otherwise, the term alkyl within the meaning of the present invention represents a linear or branched alkyl radical comprising between 1 and 12 carbon atoms such as the methyl, ethyl, propyl, isopropyl, butyl, isobutyl, sec-butyl and tent-butyl, pentyl or amyl, isopentyl, neopentyl, hexyl or isohexyl, heptyl, octyl, nonyl, decyl, undecyl or dodecyl radicals. It is understood that in the present application the alkyl radical can be of CnH2n type, and have two linking points, at the start and end of the chain (also called alkanediyl). Preferentially the alkyl radical is a (C1-C6)alkyl radical, i.e. representing an alkyl radical having 1 to 6 carbon atoms as defined above, or a (C1-C4)alkyl radical representing an alkyl radical having 1 to 4 carbon atoms such as for example the methyl, ethyl, propyl, isopropyl, butyl, isobutyl, sec-butyl and tert-butyl radicals.

The term alkyl in the expressions alkyloxy (or alkoxy), alkylamino, dialkylamino, aralkyl represents an alkyl radical as defined above.

More particularly, by alkylamino, is meant an alkyl radical at least one of the hydrogen atoms of which is replaced by an amine function, preferably, an alkyl radical is meant at least one of the terminal hydrogen atoms of which, i.e. at an alkyl chain end, is replaced by an amine function such as for example, and preferentially a —(CH2)2-NH radical, or a —(CH2)3-NH radical.

More particularly, by alkoxy, is meant an alkyl radical at least one of the terminal hydrogen atoms of which, i.e. at one end of the alkyl chain, is replaced by an oxygen atom such as for example, and preferentially a —(CH2)2-O— radical, or a —(CH2)3-O— radical.

Within the meaning of the present invention, the aryl radicals can be of aromatic mono- or polycyclic type. The monocyclic aryl radicals can be chosen from the phenyl, tolyl, xylyl, mesityl, cumenyl and preferably phenyl radicals. The polycyclic aryl radicals can be chosen from the naphthyl, anthryl, phenanthryl, fluorenyl radicals. They can be optionally substituted by one or more identical or different radicals such as alkyl, haloalkyl, alkoxy, alkoxycarbonyl, alkylcarbonyloxy, halo, cyano, nitro, aryl, aryloxy, aryloxycarbonyl, or arylcarbonyloxy.

The term aryl in the expression aralkyl represents an aryl radical as defined above. For example, by aralkyl is meant a benzyl radical.

By saturated heterocycle, unless defined otherwise, is meant a saturated carbon-containing cyclic radical comprising at least one heteroatom chosen from N, O and S, such as oxirane, aziridine, azetidine, piperidine. Preferably, the saturated heterocycle comprises from 3 to 7 members, preferably from 3 to 6 members, preferably from 4 to 6 members, more preferably from 5 to 6 members.

By diazacycloalkene compound, is meant a condensed bicyclic compound comprising 2 nitrogen atoms and at least one double bond.

A subject of the invention is also a pharmaceutical composition comprising at least one polymer according to the invention.

Unless defined otherwise, all the technical and scientific terms used in the present application have the same meaning as that commonly understood by an ordinary specialist in the field to which the invention belongs.

The following examples are given to illustrate the invention and should in no case be considered as limiting the scope of the invention.

### EXAMPLES

### Examples 1 and 2

### General Operating Method

Lactide (LA) and the aminated initiator (1 equivalent) are dissolved in freshly distilled dichloromethane ([LA]₀=1 mol·L⁻¹). The reaction medium is stirred for 20 minutes at T=26° C. (until the amine is completely incorporated, monitored by ¹H NMR spectroscopy) then DBU (0.01 equivalent) is added and the reaction medium is stirred at T=26° C., for between 3 and 10 minutes, until the lactide is completely consumed (also monitored by ¹H NMR spectroscopy).

10 equivalents (with respect to the DBU) of Amberlyst A15 resin (˜5 meq/g), washed and dried beforehand, are added in order to eliminate the catalyst. The reaction medium is stirred for 10 minutes then filtered. Another 5 equivalents of Amberlyst resin A15 are added to the reaction medium which is stirred for 10 minutes then filtered. The reaction solvent is then evaporated off under vacuum then the polymer obtained is dried under vacuum for 48 hours at 50° C. in the case of the linear polyesters and at 60° C. in the case of the star polyesters.

### Example 1

Polymer Initiated by Dodecylamine and with a DP=3.5

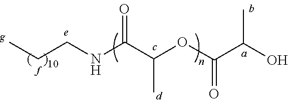

¹H NMR (δ, CDCl₃, 300.1 MHz): 6.15 (1H, br s, NH), 5.20-5.13 (6.2H, m, CH_{c}), 4.35 (1H, q, J=6.7 Hz, CHₐ), 3.20 (2H, m, CH₂ₑ), 1.60-1.54 (16H, m, CH_{3d}), 1.48 (8H, m, CH_{3d}, CH_{3b} and CH_{2f}), 1.25 (18H, m, CH_{2f}), 0.88 (3H, t, J=6.7 Hz, CH_{3g}) ppm.

¹³C NMR (δ, CDCl₃, 75.5 MHz): 169.6 (CO), 71.8 (CH) 69.8-68.5 (CH), 66.7 (CH—OH), 39.4 (CH₂N), 31.9 (CH₂), 29.7-29.6 (CH₂), 29.4-29.3 (CH₂), 26.9-26.8 (CH₂), 22.7 (CH₂), 21.4 (CH₃), 20.5 (CH₃), 17.8 (CH₃), 16.8-16.7 (CH₃), 14.1 (CH₃) ppm.

DP_{NMR}=3.6%

% amine incorporated >99%

SEC (THF): Mₙ=1056, M_{w}/Mₙ=1.14.

### Example 2

Polymer Initiated by Diethanolamine in the Presence of 6 Equivalents of D,L-Lactide

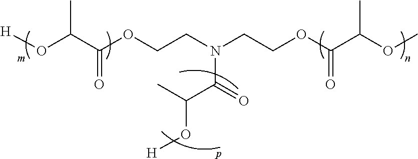

¹H NMR (δ, CDCl₃, 300.1 MHz): 5.21-5.14 (8.3H, m, CH), 4.35-4.23 (7H, m, CH—OH and CH₂—O), 2.87 (2H, m, CH₂—N), 1.60-1.50 (23, m, CH₃), 1.46-1.48 (9.1H, m, CH₃—OH) ppm.

¹³C NMR (δ, CDCl₃, 75.5 MHz): 169.6 (CO), 69.4-69.0 (CH), 66.6 (CH), 64.6 (CH₂), 47.4 (CH₂), 20.4 (CH₃), 16.6 (CH₃) ppm.

DP_{NMR}=5.7

% amine incorporated >99%

SEC (THF): Mₙ=1134, M_{w}/Mₙ=1.25.

### Example 3

Synthesis of a D,L-PLA with a DP=30 Initiated by Dodecylamine

D,L-lactide (30 equivalents) and dodecylamine (1 equivalent) are dissolved in freshly distilled dichloromethane ([LA]₀=1 mol·L⁻¹). The reaction medium is stirred for 20 minutes at ambient temperature then the DBU (0.05 equivalent) is added. The mixture is stirred vigorously at ambient temperature until the lactide is completely consumed, monitored by ¹H NMR spectroscopy. After 5 minutes, the reaction medium is neutralized by the addition of benzoic acid. The organic phase can be washed with water, then a saturated solution of NaHCO₃, and finally with a saturated solution of NaCl in order to eliminate the catalyst. The organic phase is then dried over Na₂SO₄, filtered and evaporated in order to produce the polymer.

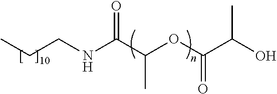

¹H NMR (δ, CDCl₃, 300.1 MHz): 6.19 (1H, br s, NH), 5.17-5.14 (57H, m, CH), 4.35 (1H, q, CH), 3.27-3.05 (2H, m, CH₂), 1.58-1.53 (178H, m, CH), 1.24 (18H, br s, CH₂), 0.87 (3H, t, CH) ppm.

DP_{NMR}=29

% amine incorporated >99%

SEC (THF): M_{w}=6608, M_{w}/Mₙ=1.18

### Example 4

Synthesis of a PLGA Copolymer 80/20 with a DP=3.5 Initiated by Dodecylamine

Lactide (2.8 equivalents), glycolide (0.7 equivalents) and dodecylamine (1 equivalent) are dissolved in freshly distilled dichloromethane ([L]₀=1 mol·L⁻¹). The reaction medium is stirred for 25 minutes at ambient temperature then the DBU (0.05 equivalent) is added and the reaction medium is stirred vigorously. After 3 minutes, the reaction medium is neutralized by the addition of benzoic acid and the total consumption of the monomer is verified by ¹H NMR spectroscopy.

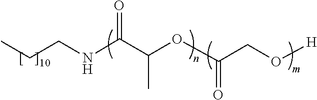

¹H NMR (δ, CDCl₃, 300.1 MHz): 6.28 (1H, br s, NH), 5.25-5.15 (4.5H, m, CHₚₒₗ), 4.90-4.67 (3.0H, m, CH₂ₚₒₗ), 4.35 (1H, m, CH), 3.32-3.21 (2H, m, CH₂), 1.60-1.48 (18.4H, m, CH₂ and CH₃), 1.24 (18h, br s, CH₂), 0.87 (3H, t, CH₃) ppm.

DP_{NMR}=3.5

% amine incorporated: >99%

Lactide/glycolide ratio=79/21 (by ¹H NMR)

SEC (THF): M_{w}=1008, M_{w}/Mₙ=1.19

### Example 5

Synthesis of an L-PLA with a DP=30 Initiated by Benzylamine

L-lactide (30 equivalents) and benzylamine (1 equivalent) are dissolved in freshly distilled dichloromethane ([LA]₀=1 mol·L⁻¹). The reaction medium is stirred for 30 minutes at ambient temperature then the DBU (0.06 equivalent) is added. The mixture is stirred vigorously. After 5 minutes, the reaction medium is neutralized by the addition of benzoic acid and the total consumption of the monomer is verified by ¹H NMR spectroscopy.

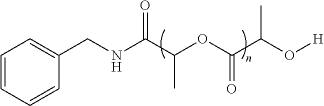

¹H NMR (δ, CDCl₃, 300.1 MHz): 7.42-7.27 (5H, m, CH), 6.62 (1H, br s, NH), 5.25-5.15 (52H, q, J=7.1 Hz, CHₚₒₗ), 4.48 (2H, m, CH₂), 4.39 (1H, q, J=6.9 Hz, CH), 1.61-1.47 (160H, m, CH₃) ppm.

¹³C NMR (δ, CDCl₃, 75.5 MHz): 169.6 (CO), 129.2 (C), 128.6 (CH), 127.7 (CH), 127.5 (CH), 71.8 (CH), 69.8 (CH), 69.0 (CH), 66.7 (CHOH), 43.2 (CH₂), 20.5 (CH₃), 17.8 (CH₃), 16.6 (CH₃ₚₒₗ) ppm. The CO amide is not observed.

DP_{NMR}=26

% amine incorporated: >99%

SEC (THF): M_{w}=8114, M_{w}/Mₙ=1.12

### Example 6

Synthesis of an L-PLA with a DP=30 Initiated by 1.3 Propanediamine

L-lactide (30 equivalents) and 1,3 propanediamine (1 equivalent) are dissolved in freshly distilled dichloromethane ([LA]₀=1 mol·L⁻¹). The reaction medium is stirred for 20 minutes at ambient temperature then the DBU (0.02 equivalent) is added and the mixture is stirred vigorously. After 3 minutes, the reaction medium is neutralized by the addition of benzoic acid and the total consumption of the monomer is verified by ¹H NMR spectroscopy.

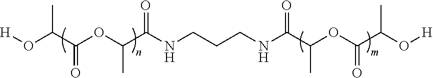

¹H NMR (δ, CDCl₃, 300.1 MHz): 6.81 (2H, br s, NH), 5.16 (63H, q, J=7.1 Hz, CHₚₒₗ), 4.37 (2H, q, J=6.9 Hz, CH), 3.48 (2H, m, CH₂), 3.25 (4H, m, CH₂), 1.58 (195H, d, J=7.1 Hz, CH₃) ppm.

¹³C NMR (δ, CDCl₃, 75.5 MHz): 169.6 (CO), 71.7 (CH), 69.1 (CH), 66.7 (CHOH), 35.5 (CH₂), 20.5 (CH₃), 17.8 (CH₃), 16.7 (CH₃ₚₒₗ) ppm. The CO amide and the central CH₂ are not observed.

DP_{NMR}=32.5

% amine incorporated: >99%

SEC (THF): M_{w}=8709, M_{w}/Mₙ=1.12

### Example 7

Synthesis of a D,L-PLA with a DP=30 Initiated by tris(2-aminoethyl)amine

D,L-lactide (30 equivalents) and tris(2-aminoethyl)amine (1 equivalent) are dissolved in freshly distilled dichloromethane ([LA]₀=1 mol·L⁻¹). The reaction medium is stirred for 30 minutes at ambient temperature then the DBU (0.1 equivalent) is added and the mixture is stirred vigorously. After 10 minutes, the reaction medium is neutralized by the addition of benzoic acid and the total consumption of the monomer is verified by ¹H NMR spectroscopy.

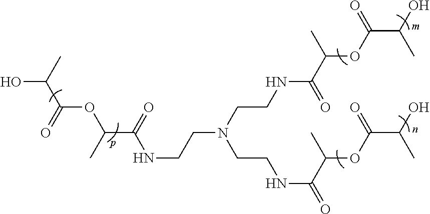

¹H NMR (δ, CDCl₃, 300.1 MHz): 6.91-6.71 (3H, br s, NH), 5.20-5.14 (72H, m, CHₚₒₗ), 4.35 (3H, q, J=6.9 Hz, CH), 3.44-3.04 (6H, m, CH₂), 2.64-2.48 (6H, m, CH₂), 1.61-1.48 (225H, m, CH₃) ppm.

¹³C NMR (δ, CDCl₃, 75.5 MHz): 175.1 (CO), 169.6 (CO), 69.1 (CH), 69.0 (CH), 66.7 (CHOH), 54.4 (CH₂), 38.0 (CH₂), 20.5 (CH₃), 17.6 (CH₃), 16.6 (CH₃) ppm.

DP_{NMR}=37.5

% amine incorporated >99%

SEC (THF): M_{w}=8514, M_{w}/Mₙ=1.09

### Example 8

Synthesis of a D,L-PLA with a DP=3.5 Initiated by N-Methyl Dodecylamine, in THF at 70° C.

D,L-lactide (3.2 equivalents) and N-methyldodecylamine (1 equivalent) are dissolved in freshly distilled THF ([LA]₀=1 mol·L⁻¹). The reaction medium is stirred for 3 hours at T=70° C. then the DBU (0.05 equivalent) is added and the mixture is stirred vigorously at ambient temperature. After 4 minutes, the reaction medium is neutralized by the addition of benzoic acid (1.5 eq, 21 mg). The THF is evaporated off and the reaction medium is taken up in 70 mL of dichloromethane. The organic phase is washed twice with a saturated solution of NaHCO₃, once with water, once with a saturated solution of salt, then dried over Na₂SO₄, filtered and evaporated. The polymer obtained is dried for 48 h at 50° C. then stored under argon.

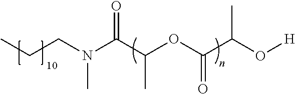

¹H NMR (δ, CDCl₃, 300.1 MHz): 5.37 (1H, m, CH), 5.20 (4.8H, m, CHₚₒₗ), 4.37 (1H, m, CH) 3.45 (0.6H, m, CH₂), 3.23 (1.4H, m, CH₂), 2.99-2.91 (3H, m, CH₃), 1.59-1.48 (22H, m, CH₃), 1.26 (18H, br s, CH₂), 0.88 (3H, m, CH₃) ppm.

¹³C NMR (δ, CDCl₃, 75.5 MHz): 175.1 (CO), 169.6 (CO), 169.4 (CO), 69.0 (CH), 67.8 (CH), 66.7 (CHOH), 49.6 (CH₂), 48.2 (CH₂), 34.7 (CH₃), 33.8 (CH₃), 31.9 (CH₂), 29.6 (CH₂), 29.5 (CH₂), 29.3 (CH₂), 28.3 (CH₂), 27.0 (CH₂), 26.8 (CH₂), 26.7 (CH₂), 22.7 (CH₂), 20.5 (CH₃), 17.2 (CH₃), 16.6 (CH₃), 16.3 (CH₃), 14.1 (CH₃) ppm.

DP_{NMR}=3.4

% amine incorporated >96%

Yield=75%

SEC (THF): M_{w}=1162, M_{w}/Mₙ=1.24

### COMPARATIVE EXAMPLES

In order to demonstrate the advantages of the method according to the invention, comparative tests were carried out.

A first polymer was synthesized starling from an alcohol initiator (1-dodecanol). The other polymers synthesized starting from an amine initiator, according to the invention, are those of Examples 1 and 8.

The following properties were obtained:

| | Average Mm | | |
|---|---|---|---|
| | (g/mol) | Tg (° C.) | Appearance |
| Comparative (ester) | 1324 | −30 | Fluid |
| Example 1 | 1372 | −25 | Pasty (crystalline) |
| (primary amine) | | | |
| Example 8 | 1067 | −36 | Fluid (Amorphous) |
| (secondary amine) | | | |

This adjustment of the properties of the polymer is not possible with a PLA/PLGA initiated with an alcohol. Initiation with the amines makes it possible to adjust the fluidity and crystallinity depending on the type of amine used. In fact, it is possible to create hydrogen bond(s) between the chains, which is not possible with alcohol initiation.

1. A method for preparing linear polymers with an amide end or a star architecture with an amide core comprising polymerizing a lactide monomer, by ring opening, comprising the steps of:
(i) reacting the monomer or monomers in excess with an initiator in a solvent, wherein said initiator is an amine or an amino alcohol, and wherein said initiator has at least one primary or secondary amine function,
(ii) adding a catalyst, wherein said catalyst is a non-nucleophilic base, and comprising at least one neutral nitrogen atom of sp2 type, and
(iii) neutralizing the reaction mixture.

2. The method according to claim 1, wherein the monomer is lactide.

3. The method according to claim 1, wherein the polymers are prepared based on a lactide monomer and a glycolide monomer.

4. The method according to claim 1, wherein step (ii) is carried out after the complete incorporation of the initiator.

5. The method according to claim 1, wherein the basic catalyst is:
1,8-diazabicyclo[5.4.0]undec-7-ene;
1,5-diazabicyclo[4.3.0]non-5-ene;
a 4-amino-pyridine compound of formula: 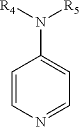
wherein R₄ and R₅ are independently chosen from a hydrogen atom or a C₁-C₁₂ alkyl radical; or
R₄ and R₅ form together with the nitrogen atom a saturated heterocycle;
a cyclic guanidine of formula: 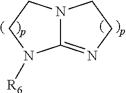
wherein p is 1 or 2, and R₆ represents a hydrogen atom or a C₁-C₄ alkyl radical;
a phosphazene of formula: 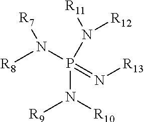
wherein R₇, R₁₀, R₁₁, and R₁₂ represent independently a C₁ to C₆ alkyl radical;
R₈ and R₉ represent independently a hydrogen atom or a C₁ to C₆ alkyl radical, or R₈ and R₉ form together with the nitrogen atoms a saturated heterocycle; and
R₁₃ represents a C₁ to C₆ alkyl radical.

6. The method according to claim 1, wherein the reaction takes place in an organic solvent.

7. The method according to claim 6, wherein the solvent is a halogenated solvent.

8. The method according to claim 1, wherein the initiator is an amine.

9. The method according to claim 1, wherein the initiator is an amino alcohol.

10. The method according to claim 1, wherein the polymers with star architecture are prepared at a reaction temperature ranging from 0 to 150° C.

11. A polymer of formula (I) 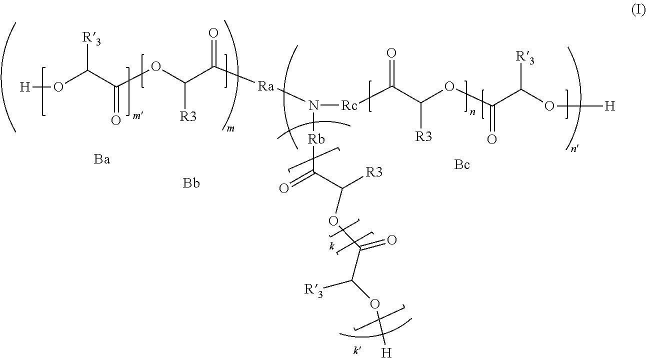
wherein
n, n′, m, m′, k and k′ represent independently an integer from 0 to 12;
Rₐ represents a covalent bond, a linear or branched C₅ to C₁₄ alkyl radical, an alkylamino radical, an alkyloxy radical, an aryl or aralkyl radical, wherein if Rₐ is an aryl or aralkyl radical then m and m′ are zero;
R_{b} represents a covalent bond, a linear or branched C₅ to C₁₄ alkyl radical, an alkylamino radical or an alkyloxy radical;
R_{c} represents a covalent bond, a linear or branched C₅ to C₁₄ alkyl radical, an alkylamino radical, or an alkyloxy radical;
R₃ represents a hydrogen atom and R′₃ represents an alkyl radical, wherein at least one of the n′, m′ and k′ is different from zero;
or R′₃ represents a hydrogen atom and R₃ represents an alkyl radical, wherein at least one of the n, m and k is different from zero;
and wherein:
at most one of the branches Bₐ, B_{b} and B_{c} represents a hydrogen atom;
if one of the branches Bₐ, B_{b} and B_{c} is a hydrogen atom, then at least one of the other two branches is linked to the nitrogen atom by an alkylamino radical.

12. The polymer according to claim 11, wherein R₃ represents an alkyl radical, and n′, m′ and k′ are zero.

13. The polymer according to claim 11, wherein one of the branches Bₐ, B_{b} and B_{c} represents a hydrogen atom.

14. The polymer according to claim 11, which wherein at least one of Rₐ, R_{b} and R_{c} represents an alkylamino radical.

15. The polymer according to claim 11, wherein at least one of Rₐ, R_{b} and R_{c} represents an alkyloxy radical.

16. A pharmaceutical composition comprising at least one polymer according to claim 11 and a pharmaceutically active agent.

17. The method according to claim 6, wherein the organic solvent is an aromatic solvent.

18. The method according to claim 7, wherein the halogenated solvent is dichloromethane.

19. The method according to claim 10, wherein the reaction temperature ranges from 20 to 45° C.

20. The method according to claim 1, wherein the lactide monomer is polymerized in the presence of a glycolide.
